(19) United States
(12) Patent Application Publication
(10) Pub. No.: US 2002/0086054 A1
(43) Pub. Date: Jul. 4, 2002

(54) Controlled release system that needs no drilling

(76) Inventor: Jiajiu Shaw, Ann Arbor, MI (US)

Correspondence Address: Jiajiu Shaw, 3166 Shamrock Ct., Ann Arbor, MI, 48105, US

(21) Appl. No.: 09/755,567

(22) Filed: Jan. 2, 2001

(51) Int. Cl. A61K 9/24

(52) U.S. Cl. 424/473

(57) ABSTRACT

Disclosed is a novel controlled release delivery system, which is a semi-permeable membrane-controlled solid dosage form. Unlike several known systems, where one or more holes are drilled on the semi-permeable membrane, the system disclosed in the present invention does not need any drilling on the semi-permeable membrane.

### BACKGROUND—FIELD OF INVENTION

This invention relates to a novel controlled release system of pharmaceutical dosage forms. Said system is a membrane-controlled delivery system, which requires no drilling,

### BACKGROUND—DESCRIPTION OF PRIOR ART

Controlled release delivery system has been of interest to many researchers in pharmaceutical industry. There are several advantages of controlled release dosage forms as compared to conventional immediate release dosage forms. For instance, a patient may need to take certain tablet three times a day. With a controlled release dosage form, the patient may need only one tablet a day. Another example of controlled release system is that enteric-coated tablets have been used to protect the active ingredient against gastric fluid and release the active ingredient only in the intestine.

Several different kinds of controlled release systems have been used in pharmaceutical industry in recent years. One of the popular systems is a membrane-controlled delivery system.

A membrane-controlled delivery system developed by Alza Corporation, often called the Oros system, has been widely used in pharmaceutical industry (U.S. Pat. No. 3,845,770 and U.S. Pat. No. 3,916,899). In a typical Oros system, one orifice (hole) is drilled on the semi-permeable membrane surrounding a tablet. Said semi-permeable membrane is permeable to water but not permeable to drug or excipients. Under an aqueous environment such as gastric fluid, water penetrates into the tablet through said semi-permeable membrane and builds up the osmotic pressure, thus, causing the drug composition to be pushed out through the hole on said membrane. In some cases, several holes are drilled on the semi-permeable membrane on one or both sides of the Oros system. Examples of products using Alza's Oros system are Tegretol®-XR by Novartis Pharmaceuticals (Physicians' Desk Reference, 54 Edition, 2000, pp. 326 and 2052) and Procardia XR® by Pratt Pharmaceuticals, Division of Pfizer (Physicians' Desk Reference, 54 Edition, 2000, pp. 331 and 2363). modified Oros system, different from the above-mentioned Oros system, also known as a “push-pull” system, was also developed by Alza Corporation (U.S. Pat. No. 5,091,190). In this system, a tablet consists of two drug compositions, which are in a two-layered format. The two layers are (1) the “drug layer” containing the active ingredient, and (2) the “push layer” containing a hydrogel. After this two-layered tablet is coated by a semi-permeable membrane, a hole is then drilled on the semi-permeable membrane on the specific side nearest the drug layer. Basically, the drug is pushed out by the combination of two driving forces: (1) the osmotic pressure within the chamber confined by the semi-permeable membrane, and (2) the expansion of the hydrogel. One representative example is Glucotrol XR® by Pratt Pharmaceuticals, Division of Pfizer) (Physicians' Desk Reference 54 Edition, pp. 331 and 2347).

Yet another modified version of the Oros system by Alza was articulated in Chemical & Engineering News (Sep. 18, 2000, page 57). This system is made of three layers, two drug layers and one push layer, and coated by a semi-permeable membrane. The system is capsule shaped, much longer than it is wide, with a hole drilled on the semi-permeable membrane on the side nearest the top drug layer.

Although the above Oros and its modified versions by Alza are all interesting, there are common disadvantages of all of these membrane controlled systems. First, each system must have one or more holes on the semi-permeable membrane for the release of the pharmaceutical ingredient or ingredients. Also, the hole is usually drilled by a laser machine or by a sophisticated mechanical drill, which is expensive. In addition, the hole or holes must be drilled on the tablet one by one and the process is very time consuming. Yet another disadvantage is that in the case of two-layered or three-layered systems, additional step is required to oriented the tablet to ensure the hole or holes is drilled on the specific side of the tablet.

### OBJECTS AND ADVANTAGES

In light of the above disadvantages of known prior art related to the Oros system or its variations, the objects and advantages of the present invention are:

1. to eliminate the use of expensive laser drill or sophisticated mechanical drill;

2. to eliminate the time needed to drill the hole or holes on the semi-permeable membrane;

3. to simplify the process in making a semi-permeable membrane-controlled release system;

4. to reduce the total manufacturing time in making the membrane-controlled release system;

5. to reduce the total cost in producing a membrane-controlled release system;

6. to provide a semi-permeable membrane-controlled delivery system, which can have different kinds of release patterns; and

7. to provide a semi-permeable membrane-controlled release system, which can be made by standard pharmaceutical technique.

Further objects and advantages are to provide a membrane-controlled release system, having a multi-layered tablet, without having to pick a specific side of the tablet and without drilling on the specific side of a tablet. Still further objects and advantages will become apparent from a consideration of the ensuing description.

### DETAILED DESCRIPTION OF THE INVENTION

Membrane-controlled release system plays an important role in pharmaceutical industry. Several inventions in this area have been published. In the present invention, a significantly improved Oros system is disclosed. The delivery system in the present invention provides significant improvement on the currently known delivery systems.

The present invention provides a novel controlled release delivery system, which is a semi-permeable membrane-controlled solid dosage form. Unlike Oros system or its known variations, where one or more holes are drilled on the semi-permeable membrane, the system disclosed in the present invention does not need any drilling on the semi-permeable membrane.

Yet another aspect of the delivery system in this invention is that it can be made by standard pharmaceutical technology and does not need an expensive laser drill or other sophisticated mechanical drill, thereby, saving a lot of time and costs.

Basically, the delivery system disclosed in the present invention comprises the following:

(A) a tablet with one or a plurality of predetermined indentations on the surface of said tablet, and

(B) said tablet from (A) is coated with a semi-permeable membrane with a predetermined thickness.

Said membrane is significantly thinner in the proximity of said indentation due to the shape and the depth of said indentation as well as the nature of a standard coating process, whereby when said delivery system is in an aqueous environment, osmotic pressure within the chamber confined by said semi-permeable membrane causes the membrane to break up at said indentation and creates a hole on said semi-permeable membrane, thus, releasing the ingredients through this newly created hole.

Said tablet comprises a drug composition comprising one or more of the following: active ingredient, osmotic agent, excipients, hydrogel, and the combination thereof

Said indentation on the tablet has a predetermined geometric shape and depth so that when the semi-permeable membrane is coated onto the tablet by a standard coating technique, the membrane in the proximity of the indentation is significantly thinner than that on the other surface of the tablet. The thin area in the proximity of said indentation is due to the shape and the depth of said indentation as well as the nature of the coating process. The proximity of said indentation includes the bottom, the wall, the edge of the indentation, or the combination thereof.

Said indentation on the tablet is made during a standard tablet compressing process employing a standard tablet punch. Said tablet punch has a predetermined emboss so that an indentation, having a predetermined geometric shape and depth, can be made on said tablet.

Said geometric shape comprises one or a plurality of straight or curved line segments. Said geometric shape includes, but not limited to, “−”, “+”, “*”, “>”, “˜”, “#”, “=”, “$”, “&”, “(”, “@”, “A”, “B”, “C”, “D”, “E”, “H”, “I”, “L”, “M”, “N”, “P”, “S”, “T”, “U”, “V”, “W”, “X”, “Y”, or “Z”.

Said geometric shape may also be a polygon including, but not limited to, triangle, square, pentagon, hexagon, heptagon, octagon, parallelogram, or trapezoid.

Said tablet can be a two-layered tablet with a drug layer and a push layer. The drug layer comprises one or a plurality of active ingredients and the push layer comprises one or a plurality of hydrogels. In this case, the indentation is on the drug-layer side. Said indentation on said two-layered tablet is made during a standard tablet compressing process employing a standard tablet punch. Said tablet punch has a predetermined emboss so that an indentation, having a predetermined geometric shape and depth, can be made on said two-layered tablet. After said two-layered tablet is compressed, it is then coated by a semi-permeable membrane by a standard coating technique. This is different from Alza's two-layered system wherein the hole is drilled on a specific side of the tablet after being coated by a semi-permeable membrane. Therefore, the system in the present invention can save a significant time and efforts compared to Oros system or its modified version.

Said semi-permeable membrane comprises a pharmaceutically acceptable polymer, polymer blend, or the combination thereof Said polymer includes, but not limited to, cellulose ester, cellulose ether, cellulose ester-ether, acrylic acid polymer, acrylic acid co-polymer, polyvinyl acetate, silicon polymer, or the combination thereof.

Said system in the present invention may have a smaller sub-tablet embedded in the tablet. Said sub-tablet comprises a drug composition comprising the following: active ingredient, osmotic agent, pharmaceutically acceptable excipients, hydrogel, and the combination thereof The sub-tablet may be coated by a permeable or semipermeable membrane, which comprises a pharmaceutically acceptable polymer, polymer blend, or the combination thereof Said polymer includes, but not limited to, cellulose ester, cellulose ether, cellulose ester-ether, acrylic acid polymer, acrylic acid co-polymer, polyvinyl acetate, silicon polymer, or the combination thereof

Said tablet of the system in the present invention may also have a plurality of pellets, which are either coated or un-coated. The pellets comprise a drug composition comprising the following: active ingredient, osmotic agent, pharmaceutically acceptable excipients, hydrogel, or the combination thereof When the pellets are coated, the coating membrane comprises a pharmaceutically acceptable polymer, polymer blend, excipients, or the combination thereof, and the membrane may be semi-permeable or permeable.

Said semi-permeable membrane comprises a pharmaceutically acceptable polymer, polymer blend, or the combination thereof Said polymer includes, but not limited to, cellulose ester, cellulose ether, cellulose ester-ether, acrylic acid polymer, acrylic acid co-polymer, polyvinyl acetate, silicon polymer, or the combination thereof.

Said permeable membrane on said sub-tablet or said pellet comprises a pharmaceutically acceptable polymer with or without pore-generating agents. Examples of said polymers include, but not limited to cellulose ester, cellulose ether, cellulose ester-ether, acrylic acid polymer, acrylic acid co-polymer, polyvinyl acetate, silicon polymer, or the combination thereof Said pore-generating agents includes pharmaceutically acceptable water soluble ingredients including, but not limited to, ethyleneglycol, cellulose, lactose, glucose, fructose, sucrose, dextrose, maleic acid, fumaric acid, mannitol, sodium chloride, or citric acid.

An erodible outer layer may be coated on said semi-permeable membrane on said tablet. This overcoat comprises an active ingredient, hydrogel, surfactant, pharmaceutical excipients, or the combination thereof.

In the system of the present invention, the basic mechanisms for the release of the desired drug composition may be illustrated as follows. However, other mechanisms may also be involved. The mechanisms presented below are only representations and do not limit the present invention.

### (A) The Role of the Indentation

The indentation on the tablet has a predetermined geometric shape and depth so that the coated membrane in the proximity of the indentation is thinner than that on other surface of the tablet. Due to this indentation on the tablet surface, the weakest point of the whole membrane is in the proximity of the indentation. The weakest point may be at the bottom, the wall, or edge of said indentation. Different geometric shape, size, and depth may be used to achieve the desired results. The weakest point on the semi-permeable membrane at the indentation makes it the easiest to break by the osmotic pressure built up in the chamber confined by the semi-permeable membrane.

### (B) The Role of the Semi-permeable Membrane on the Tablet

The semi-permeable membrane serves as the primary control for the release of the desired drug composition. Imbibing fluid through the semi-permeable membrane into the chamber confined by the semi-permeable membrane causes the increase of the osmotic pressure, which subsequently breaks up the semi-permeable membrane in the proximity of the indentation and creates a hole. Through this hole, ingredients confined in the semi-permeable membrane can be slowly released mainly by osmotic pressure.

### (C) The Role of the Smaller Sub-tablet Embedded in the Tablet

(1) When the Sub-tablet Comprises an Active Ingredient

During the release process, the concentration of the active ingredient in the tablet is gradually reduced. At a later stage, the concentration of the active ingredient in the tablet becomes significantly lower than it was originally. At this time, the active ingredient in the sub-tablet starts to be dissolved, thus, replenishing the osmotic pressure in the whole system.

(2) When the Sub-tablet Comprises a Hydrogel

Similar to the situation in (1), at a later stage of the release process, the all. hydrogel in the sub-tablet starts to imbibe a significant amount of water and expand. The expansion of the hydrogel becomes an additional driving force to deliver drug composition out of the system through the hole on the semi-permeable membrane.

(3) When the Sub-tablet Comprises Water-soluble Excipients

Similar to the above-mentioned situations, at a later stage of the release process, the water-soluble excipients starts to imbibe a significant amount of water and functions as a secondary source of osmotic pressure, thereby, prolonging the drug delivery process.

### (D) The Role of the Pellets

Similar to the situations in (C), pellets can have similar functions as the sub-tablet.

### (E) The Role of the Membrane on the Sub-tablet or Pellets

The membrane on the sub-tablet or the pellets serves as an additional control for releasing the ingredients within the sub-tablet or the pellets. It may also serve for constraining the sub-tablet or pellets so that when the sub-tablet or the pellets expand, one or more smaller “balloons” are created within the semi-permeable membrane of the tablet. These small balloons can then serve as small pushing devices to help prolong the drug delivery process.

### (F) The Role of the Optional Erodible Overcoat on the Whole System

When the overcoat comprises the active ingredient, whether it is the same as or different from that in the tablet, the system disclosed in the present invention can release the active ingredient in an immediate-release manner, such as a bolus dose. When the overcoat does not contain an active ingredient, it can function as a delayed-release mechanism. The overcoat may also serve for a cosmetic reason, such as a desired color.

Some representative examples of the system in the present invention are listed as follow:

### EXAMPLE 1

A pharmaceutical tablet is made by standard pharmaceutical method having a predetermined size of indentation in “+” shape; the edge of the indentation has an angle of about 90 degree; the depth of the indentation is about 1.0 mm. The tablet is then coated by a semi-permeable polymer, such as polyvinyl acetate, with a predetermined thickness. The thickness of the semi-permeable membrane is generally even on the surface of the tablet except at the indentation where the membrane thickness is thinner at the bottom, the wall, or the edge of the indentation. Under an aqueous environment, the semi-permeable membrane will break up and create a hole at the proximity of the indentation due to the osmotic pressure, thus, allowing the drug composition within the confinement of the semi-permeable membrane to be released through the newly created hole.

### EXAMPLE 2

The coated tablet in Example 1 is over-coated with a predetermined thickness of overcoat by hydroxypropyl methylcellulose (HPMC) employing standard pharmaceutical techniques. Under aqueous environment, this HPMC overcoat gradually disintegrates, thus, delaying the creation of the hole on the semi-permeable membrane at the indentation. As a result, this overcoat creates a lag time before the system starts to release the ingredients within the semi-permeable membrane.

### EXAMPLE 3

The coated tablet in Example 1 is over-coated with a drug composition, which consists of an active ingredient as well as HPMC, surfactant, or excipients. This overcoat may serve as a source for the release of the active ingredient before the ingredients within the membrane start to be released.

### EXAMPLE 4

The coated tablet in Example 1 is over-coated with an enteric coating material normally used in pharmaceutical dosage forms. HPMC or surfactant may be added in the coating material.

### EXAMPLE 5

Using a standard pharmaceutical procedure, a smaller sub-tablet is embedded in the tablet in the pharmaceutical delivery system as described in Example 1. The sub-tablet contains polyethylene oxide as well as other suitable pharmaceutical additives. Said polyethylene oxide has a molecular weight range of 1,000,000 to 10,000,000.

### EXAMPLE 6

Using a standard pharmaceutical procedure, a pharmaceutical delivery system same as that described in Example 5 is made, except in this example, the sub-tablet comprises a pharmaceutical active ingredient. Said active ingredient is the same as that in the tablet, but with a higher concentration than that in the tablet.

### EXAMPLE 7

Using a standard pharmaceutical procedure, a pharmaceutical delivery system same as described in Example 6 is made, except in this example, the sub-tablet comprises a pharmaceutical active ingredient different from that in the tablet.

### EXAMPLE 8

Using a standard pharmaceutical procedure, a pharmaceutical delivery system as described in Example 5 is made, except in this example, the sub-tablet is coated by a semi-permeable membrane, such as polyvinyl acetate.

### EXAMPLE 9

Using a standard pharmaceutical procedure, a pharmaceutical delivery system as described in Example 1 is made, except in this example, the indentation is in “−” shape, the depth of the indentation is about 0.8 mm, and the edge between of the indentation and the surface of the tablet has an angle of about 90 degree.

### EXAMPLE 10

Using a standard pharmaceutical procedure, a pharmaceutical delivery system as described in Example 1 is made, except in this example, the indentation is a tetrahedron with one opening-side on the tablet surface and the other three sides indented into the tablet.

### EXAMPLE 11

Using a standard pharmaceutical procedure, a pharmaceutical delivery system as described in Example 1 is made, except in this example, the indentation is in a square shape, the depth is about 0.9 mm, and the edge of the indentation has an angle of about 90 degree.

### EXAMPLE 12

Using a standard pharmaceutical procedure, a pharmaceutical delivery system as described in Example 1 is made, except in this example, there are two indentations, one on each side of the tablet.

### EXAMPLE 13

Using a standard pharmaceutical procedure, a pharmaceutical delivery system as described in Example 1 is made, except in this example, there are four indentations, two on each side of the tablet.

### EXAMPLE 14

Using a standard pharmaceutical procedure, a pharmaceutical delivery system as described in Example 1 is made, except in this example, there are about 20-50 pellets in the tablet. Said pellets comprise a pharmaceutical active ingredient same as that in the tablet, but the concentration of the active ingredient is higher than that in the tablet.

### EXAMPLE 15

Using a standard pharmaceutical procedure, a pharmaceutical delivery system as described in Example 14 is made, except in this example, the active ingredient in the pellets is different from the active ingredient in the tablet.

### EXAMPLE 16

Using a standard pharmaceutical procedure, a pharmaceutical delivery system as described in Example 14 is made, except in this example, the pellets comprise hydrogel and other suitable pharmaceutical excipients, but does not contain any pharmaceutically active ingredient. These pellets can absorb water and the volume of each pellet can expand in a later stage and function as a secondary driving force to help “push” the drug composition out of the system.

### EXAMPLE 17

Using a standard pharmaceutical procedure, a pharmaceutical delivery system as described in Example 16 is made, except in this example, the pellets are coated by pharmaceutically acceptable semi-permeable membrane, such as polyvinyl acetate. Said semi-permeable membrane on the pellets help makes it easier in handling the pellets and also to separate the contents of the pellets from those of the tables in the tablet compressing process.

### EXAMPLE 18

Using a standard pharmaceutical procedure, a pharmaceutical delivery system as described in Example 14 is made, except in this example, the pellets comprise osmotic agent or agents, such as citric acid, sodium chloride, or lactose.

### EXAMPLE 19

Using a standard pharmaceutical procedure, a pharmaceutical delivery system as described in Example 18 is made, except in this example, the pellets are coated by semi-permeable membrane such as polyvinyl acetate.

### EXAMPLE 20

Using a standard pharmaceutical procedure, a pharmaceutical delivery system as described in Example 1 is made, except the following difference:

(1) The tablet has two layers, a drug layer and a push layer. Said drug layer comprises an active ingredient and suitable pharmaceutical ingredients. Said push layer comprises a hydrogel, such as polyethylene oxide, as well as other suitable pharmaceutical additives.

(2) The indentation is made on the drug layer side, not on the push layer side, during standard tablet compressing process.

Although the above description and examples contain many specificities, these should not be construed as limiting the scope of the invention but as merely providing the illustrations of some of the presently preferred embodiments of this invention.

Thus, the scope of this invention should be determined by the appended claims and their legal equivalents, rather by the examples given.

1. A semi-permeable membrane-controlled pharmaceutical delivery system that requires no drilling.

2. The delivery system of claim 1 comprising the following:
(A) a tablet with one or a plurality of predetermined indentations on the surface of said tablet, and
(B) said tablet from (A) is coated with a semi-permeable membrane with a predetermined thickness,
wherein said membrane is significantly thinner in the proximity of said indentation due to the shape and the depth of said indentation as well as the nature of a standard coating process, whereby when said delivery system is in an aqueous environment, osmotic pressure within the chamber confined by said semi-permeable membrane causes the membrane to break up at said indentation and creates a hole on said semi-permeable membrane, thus, releasing the ingredients through this newly created hole.

3. The delivery system of claim 2 wherein said tablet comprises a drug composition comprising the following: active ingredient, osmotic agent, excipients, hydrogel, or the combination thereof.

4. The delivery system of claim 2 wherein a smaller sub-tablet is embedded in said tablet wherein said sub-tablet comprises a drug composition comprising active ingredient, osmotic agent, hydrogel, excipients, or the combination thereof.

5. The delivery system of claim 4 wherein said hydrogel is pharmaceutically acceptable polyethylene oxide having a molecular weight range of 1,000,000 to 10,000,000.

6. The delivery system of claim 4, wherein said sub-tablet is coated by a pharmaceutically acceptable micro-porous membrane, wherein said micro-porous membrane is semi-permeable or permeable.

7. The delivery system of claim 2, wherein said semi-permeable membrane comprises pharmaceutically acceptable polymer, polymer blend, or the combination thereof.

8. The delivery system of claim 7 wherein said pharmaceutically acceptable polymer comprises polyvinyl acetate, cellulose ester, cellulose ether, cellulose ester-ether, acrylic acid polymer, acrylic acid co-polymer, polyvinyl acetate, silicon polymer, or the combination thereof.

9. The delivery system of claim 2, wherein a plurality of pellets are embedded in said tablet whereas said pellets comprise active ingredient, osmotic agent, excipients, hydrogel, or the combination thereof.

10. The delivery system of claim 9, wherein the pellets are coated by pharmaceutically acceptable membrane.

11. The delivery system of claim 2, wherein said indentation has a predetermined geometric shape and depth.

12. The delivery system of claim 11 wherein said geometric shape is a pattern made by one or a plurality of straight-line segments or curved line segments.

13. The delivery system of claim 12 wherein the geometric shape is “−”, “+”, “*”, “>”, “˜”, “#”, “=”, “$”, “&”, “(”, “@”, “A”, “B”, “C”, “D”, “E”, “H”, “I”, “L”, “M”, “N”, “P”, “S”, “T”, “U”, “V”, “W”, “X”, “Y”, or “Z”.

14. The delivery system of claim 13 wherein the geometric shape is a polygon, such as triangle, square, pentagon, hexagon, heptagon, octagon, parallelogram, or trapezoid.

15. The delivery system of claim 2, wherein an outer layer of membrane is coated on said semi-permeable membrane.

16. The delivery system of claim 15 wherein said outer layer comprises enteric coating material normally used in pharmaceutical dosage forms.

17. The delivery system of claim 15 wherein said outer layer comprises pharmaceutically acceptable active ingredient, excipients, or the combination thereof.

18. The delivery system of claim 2 wherein said tablet is two-layered comprising a drug layer and a push layer.

19. The delivery system of claim 18 wherein said drug layer comprises an active ingredient and suitable pharmaceutical ingredient and said push layer comprises pharmaceutically acceptable hydrogel, excipients, or the combination thereof.

20. The delivery system of claim 3, wherein the drug composition in the tablet comprises a plurality of different active ingredients.
